(19) United States
(12) Patent Application Publication
(10) Pub. No.: US 2006/0122696 A1
(43) Pub. Date: Jun. 8, 2006

(54) Methods and apparatus for in vivo cell therapy

(76) Inventors: Ping Ye Zhang, San Diego, CA (US); XiuMing Yang, San Diego, CA (US)

Correspondence Address: Ping Ye Zhang, 11865 Aspen View Drive, San Diego, CA, 92128, US

(21) Appl. No.: 11/283,326

(22) Filed: Nov. 18, 2005

Related U.S. Application Data
(60) Provisional application No. 60/629,709, filed on Nov. 22, 2004.

(51) Int. Cl. A61F 2/06

(52) U.S. Cl. 623/1.41; 424/422

(57) ABSTRACT

The present invention includes a method and apparatus to transplant functional cells into failing heart muscle to cure heart disease. In particular, the present invention relates to a method and apparatus to deliver cell-composed medical device and/or three-dimensional cell composite per minimally invasive intracornonary approach to the infarct-related artery, allowing functional cells to home in and engraft to the zone of the infarct and peri-infarct tissue, in order to regenerate infarcted, scarred or non-functioning myocardial tissue into functioning muscle (“myogenesis”), also to create growth and proliferation of new blood vessel (“angiogenesis”) in the area.

### RELATED APPLICATION

The present application claims priority to U.S. provisional patent application, entitled METHOD AND APPARATUS FOR IN VIVO CELL THERAPY, assigned application No. 60/629,709, and filed Nov. 22, 2004.

### FIELD OF THE INVENTION

The present invention relates in general to the field of methods and apparatus to transplant functional cells into failing heart muscle to cure heart disease. In particular, the present invention relates to a method and apparatus to deliver cell-composed medical device and/or three-dimensional cell composite per minimally invasive intracornonary approach to the infarct-related artery, allowing functional cells to home in and engraft to the zone of the infarct and peri-infarct tissue, in order to regenerate infarcted, scarred or non-functioning myocardial tissue into functioning muscle (“myogenesis”), also to create growth and proliferation of new blood vessel (“angiogenesis”) ,in the area.

### DESCRIPTION OF RELATED ART

When heart muscle is damaged by injury such as a heart attack, functional contracting heart muscle dies and is replaced with nonfunctional scar tissues. Heart attacks cause massive loss of heart muscle cells, known as cardiomyocytes, resulting in a diminished heart pumping ability. About 1.5 million Americans suffer a heart attack each year, which is the primary cause of heart muscle damage. Heart muscle can also be damaged by coronary artery disease. Coronary artery disease leads to the episodes of cardiac ischemia, in which the heart muscle is not getting enough oxygen-rich blood. Eventually, the heart muscle enlarges from the additional work it must do in the absence of enough oxygen-rich blood, leading to ischemic cardiomyopathy—a type of heart disease in which the heart is abnormally enlarged, thickened and/or stiffened. As a result, the heart muscle's ability to pump blood is usually weakened. This damage commonly results in congestive hear failure (“CHF”). All therapies available today for CHF are palliative (they only treat the symptoms as opposed to the underlying cause of the disease) and have modest effects on limiting the disease progression of the disease. Examples of these palliative therapies in common use today include, artificial left ventricle assist devices, biventricular pacemakers, heart transplants, and drug therapies.

In general, stem and progenitor cells are naturally occurring, self-renewing and undifferentiated primitive cells that develop into any of a number of functional, differentiated cells. For example, human embryonic stem cells are pluripotent: that is, they can develop into all cells and tissues in the body and thereby perform a specialized function. (Bishop, A., et al 2002. Embryonic stem cells. J Pathol 2002: 197: 424-429.) There are various types of human stem cells, also called hSCs: (1) human embryonic stem cells (hES), which are derived from donated in vitro fertilized blastocysts or very early-stage embryos; (2) human embryonic germ cells (hEG), which are derived from donated fetal material; (3) human embryonic carcinomas cells (hEC) derived from embryonal carcinomas; (4) adult stem cells (hS), which are derived from tissues such as bone marrow, cord blood, peripheral blood and hematopoinetic stem cell derived; (5) Skeletal myoblasts; (6) Multipotent adult progenitor cells that can differentiated into cardiomyocyte and/or endothelial cells; (7) Adult mesenchymal stem cells; and (8) other sources such as adult neuronal stem cells, hepatic stem cells, endothelial progenitor cells, adult and neonatal heart cardiomyocyte and/or non cardiomyocytes, stem cell derived from adipose (fat) tissue, skin progenitor cells.

For the emerging stem cell therapies (skeletal myoblasts, cardiomyocyotes, cardiac stem cells, endothelial progenitor cells, embryonic stem cells, adult mesenchymal stem cells), a bolus of cells is injected locally to the desired sites by delivery modes such as intramyocardial, intracoronary and intravenous (Lee, M., et al 2004. Stem Cell Transplantation in Myocardial Infarction. Review in Cardiovascular Medicine 2004: Vol 5 No. 2, 82-98.) The direct myocardial injection though is a simple process and allows for direct visualization and surveillance of the target zones, however, the surgical approach is associated with well-known operative risks of an open-heart operation. Stem cells can also be injected percutaneously with catheter-based myocardial injection guided by left ventricular electromechanical mapping with the NOGA system (Biosesnse Webster, Diamond Bar, Calif.). Cells may be directly injected into nonviable areas of the myocardium with an injection-needle catheter. Intracoronary delivery of stem cells appears to be superior to direct intramyocardial and intravenous administration in clinical practice, still-unresolved issues of intracoronary delivery stem cells include the optimum number of cells and the optimal duration of the infusion, as they may adversely affect coronary perfusion and induce myonecrosis. The above stem cell delivery modes represent drawbacks such as: (i) whether the cells are delivered in medium alone or with a polymerization agent, an optimum distribution and survival of initial cells deposits are not achieved. Cells injected or flown into scar tissue naturally migrate away from the targeted site followed by apoptosis, (ii) additionally, the transplanted cells that do take hold at the area of interest may not survive without appropriate conditions, for instance, cardiomyocyte is not resistant to ischemia.

Thus, there is a need in the art to develop methods and apparatus which are useful for delivering a sufficiently confluent population of cells to infracted, scarred or non-functioning myocardial tissue, providing conditions allowing cells to home in and engraft in the target area, preferably through minimally invasive methods to the host, in order to regenerate the targeted tissues into functioning muscle. Such improvements in implanting and localizing cell transplants will prove beneficial to numerous transplant and grafting procedures, including, but not limited to cell transplant in the heart to repair and/or replace failing heart muscle. The present invention satisfies these needs.

### BRIEF SUMMARY OF THE INVENTION

The present invention is directed to a method and apparatus for transplanting cells into a specific area, such as the heart. In one embodiment, the present invention system includes a cell-composed medical device comprising a supporting structure that is specifically-profile covered or emerged with a cell growth matrix. The supporting structure configurations are comprises of a series of overall shapes including flexible cylinders or scaffolds, referred to as stents known in the art. The supporting structure design has specific patterns to facilitate cell growth and to provide the desired lumen opening or structure integrity lost by the damaged vessels. Materials for constructing the supporting structure are well known in the art and include metal and polymer materials. Placement of the cell-composed medical device into a targeted infarct-related artery site is done via percutaneous delivery catheter. After the cell-composed medical device reaches the targeted site, the cell-composed medical device can be either balloon expandable or self-expandable to be placed in the infarct-related artery. The cells from the cell-composed medical device, home in and engraft to the zone of the infarct homogenously to create myogenesis and angiogenesis, thus to sufficiently repopulate the heart muscle, and in turn restore the heart pumping function.

In an alternative embodiment, the present invention includes three-dimensional cell composite. The said composite can be in any appropriate shapes/sizes that contain a mixture of cells, biological and nonbiological materials. Different from cell dispensable solution approach in related art, the present invention of the said three-dimensional cell composite provides conditions that will assist cells to home in and engraft in infarcted, scarred or non-functioning myocardial tissue. The said three-dimensional cell composite can be delivered to the infarct-related artery accomplished through intracoronary injection. Injection pressure can be applied to move the said cell growth composite deep into the infracted area. The cells from the said three-dimensional cell composite will home in and engraft to the zone of the infarct homogenously to create myogenesis and angiogenesis, thus to sufficiently repopulate the heart muscle, and in turn restore the heart pumping function.

### BRIEF DESCRIPTION OF THE DRAWINGS

The present invention is best understood by referring to the following description and accompanying drawings that are used to illustrate of the invention. In the drawings:

FIG. 1 is a perspective view of cell-composed medical device coated with cells and cell culture medium.

FIG. 2 is a perspective view of the embodiment of FIG. 1 delivered via percutaneous catheter into infarct-related artery.

FIG. 3 is a perspective view of three-dimensional cell composite injected to the infarct-related artery with catheter-based system.

### DETAILED DESCRIPTION OF CERTAIN EMBODIMENTS OF THE INVENTION

The present invention is directed to methods and apparatus for transplanting functional cells into failing heart muscle to cure heart disease. While the present invention is described in detail as applied to heart, those of ordinary skill in the art will appreciate that the present invention can be applied to other organs/sites.

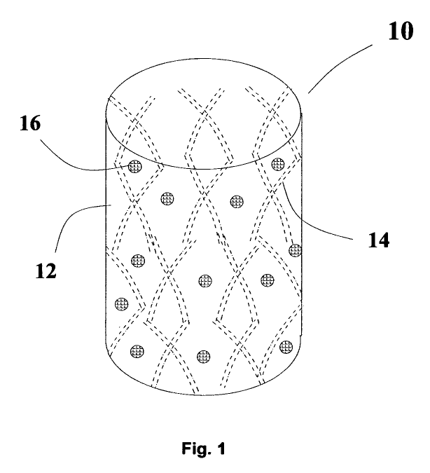

FIG. 1 illustrates a general example of cell-composed medical device to one embodiment of the present invention. The cell-composed medical device 10 comprises of cell growth matrix 12 and supporting structure 14. Cells in appropriate culture medium 16 are grown in matrix 12. Cell growth matrix 12 is attached to supporting structure 14. Supporting structure 14 is expandable and/or deformable, such as stents. Cells can grow, differentiate and migrate in matrix 12 as necessary prior to cell-composed medical device 10 delivered to the infarct-related artery.

Sources of cells could be from human embryonic stem cells (hES), human embryonic germ (hEG) cells, human embryonic carcinomas cells (hEC) and adult stem cells including, but not limited to bone marrow cells. Other genetically modified cells that give rise to any cell type in the heart, including cardimyocyte and vascular endothelial cells can also be used. Cultured immortalized cells including but not limited to satellite cells and myoblasts can also be used. Stem cells and other cells can be modified by gene transfer using means well known in the art, for example chemical transfection, biological transfection or viral infection. Cell populations are purified using a variety of well known techniques including fluorescence-activating cell sorting (FACS) or magnetic-activated cell sorting (MACS), and resistant gene selections. (See generally, Robinson J. P. Handbook of Flow Cytometry Methods, Wiley-Liss, New York, 1993; Shapiro H. Practical Flow Cytometry, Third Edition, Alan R. Liss, New York, N.Y., 1994.) In order to facilitate cell growth, differentiation and migration on the cell growth matrix 12, biological and nonbiological cell culture medium 16 can be incorporated into the matrix 12. These approaches include but are not limited to use of growth factors, such as stem cell factor, granulocyte colony-stimulating factor, stromal cell-derived factor-1, platelet derived growth factor, vascular endothelial growth factor (“VEGF”), insulin growth factor, cell adhesive molecules (e.g. Collagen, Gelatin, Laminin, Fibronectin), enzymes and small molecules, also genetic materials such as cDNA for growth factors, transcriptional factors (e.g., myogenic factors or homeodomains) and cytokines. In addition to growth factors and genetic material useful for cell growth, differentiation and migration, molecules such as rapamycin or FK560 may be added to help avoid immunorejection. As a result of the above approaches, the matrix will contain millions of functional cells, once implanted in target tissue, ready to home in and engraft in the area of interest.

Materials for constructing said cell growth matrix 12 are well known in the art and include bio-absorbable materials such as poly lactic acid (PLA), polyglycolic acid (PGA), polysebacic acid (PSA), poly(lactic-co-glycolic) acid copolymer (PLGA), poly(lactic-co-sebacic) acid copolymer (PLSA), poly(glycolic-co-sebacic) acid copolymer (PGSA), polyesters, polyorthoesters, polyanhydrides, polyiminocarbonates, hydrogel, a biodegradable polyester amide (PEA), polyphosphoester polymer, p(DAPG-EOP), in the form of micromatrixs, inorganic calcium phosphate, aliphatic polycarbonates, polyphosphazenes, collagen based adhesive, fibrin based adhesive, albumin based adhesive, polymers or copolymers of caprolactones, amides, amino acids, acetals, cyanoacrylates, degradable urethanes; or biocompatible but non-bioabsorbable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina). The cell growth matrix 12 may be made of natural materials such as Extracellular Matrix (ECM); or the individual component of the ECM including Collagen and Laminin. All of these materials have been used clinically in human for tissue repair and replacement. The above disclosure is not an exhaustive list, but instead represents alternate embodiments illustrated by way of example only.

The supporting structure 14 may be fabricated from stainless steel, platinum, rhodium, rhenium, palladium, tungsten, nitinol and the like, as well as alloys of these metals. The supporting structure 14 may be made of radiolucent fibers or polymers such as Dacron (polyester), polyglycolic acid, polylactic acid, fluoropolymers, nylon, or even silk. The supporting structure may be made of various combinations of metals and fibers to achieve desirable strength and flexibility. Those of ordinary skill in the art are knowledgeable of and will readily employ the numerous materials in the art in order to achieve the spirit of the current invention.

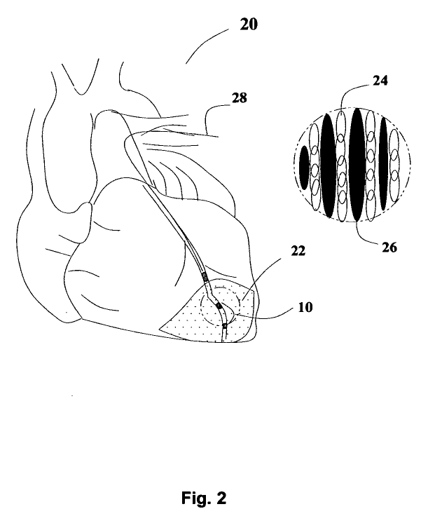

FIG. 2 illustrates perspective view of the embodiment of FIG. 1 placed into infarcted, scarred or non-functioning myocardial tissue area 22 of heart 20. Preferably, one or more cell-composed medical device 10 is delivered to the infarct-related artery through percutaneous transluminal intracoronary catheter 28. The cell-composed medical device 10 can be either balloon expandable or self-expandable to be placed around the border zone of the infarction and/or in the zone of infarction. The cells from the cell-composed medical device 10, home in and engraft to the zone of infarcted, scarred or non-functioning myocardial tissue 22 homogenously to create myogenesis 24 and new blood vessels 26 (“angiogenesis”), thus to sufficiently repopulate the heart muscle, and in turn restore the heart pumping function.

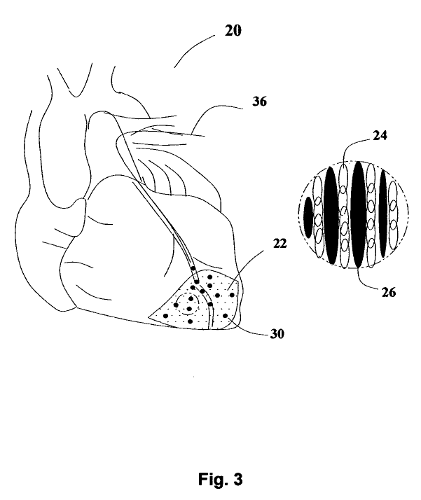

FIG. 3 illustrates perspective view of three-dimensional cell composite placed into infarcted, scarred or non-functioning myocardial tissue area 22 of heart 20. The said three-dimensional cell composite 30 contains cells, cell culture medium containing all the nutrients including growth factors, small molecules, natural occurring and/or synthetic biocompatible, biodegradable materials. Preferably, delivery of three-dimensional cell composite 30 can be accomplished through selective intracoronary injection to the infarct-related artery with catheter-based system 36. Under appropriate injection pressure, said three-dimensional cell composite 30 flow into the zone of the infarct, home in and engraft to the zone of infarcted, scarred or non-functioning myocardial tissue 22 homogenously to create myogenesis 24 and angiogenesis 26, thus to sufficiently repopulate the heart muscle, and in turn restore the heart pumping function.

What is claimed is:

1. A cell-composed medical device comprising:
functional cells;
cell culture medium;
cell growth matrix; and
supporting structure.

2. The cell-composed medical device of claim 1, wherein the functional cells, cell culture medium, the cell growth matrix and the supporting structure are compatible with the target location of a mammalian body.

3. The cell-composed medical device of claim 1, wherein the functional cells are selected from the group consisting of stem cells and progenitor cells such as human embryonic stem cells (hES); human embryonic germ cells (hEG); human embryonic carcinomas cells (hEC); adult stem cells (hS) originated from tissues including but not limited to bone marrow, peripheral blood, skeletal muscle; cornea and retina, brain, dental pulp, liver, gastrointestinal tract lining, adipose (fat) tissues and pancreas; or genetic or chemical engineered cells; or multipotent adult progenitor cells that can differentiated into cardiomyocyte and/or endothelial cells.

4. The cell-composed medical device of claim 1, wherein cell culture medium are selected from group consisting of various growth factors, enzymes, small molecules and other natural occurring and/or synthetic biocompatible materials useful for cell growth, differentiation and migration. In addition to growth factors and genetic material useful for cell growth, differentiation and migration, molecules such as rapamycin or FK560 may be added to help avoid immunorejection.

5. The cell-composed medical device of claim 1, wherein the cell growth matrix is selected from the group consisting biocompatible and biodegradable materials such as poly lactic acid (PLA), polyesters, polyester urethane (PEU), a biodegradable polyester amide (PEA); or biocompatible but non-biodegradable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina); or natural materials such as Extracellular Matrix (ECM) or the individual component of the ECM including Collagen and Laminin.

6. The cell-composed medical device of claim 1, wherein the supporting structure is selected from the group of stainless steel, platinum, rhodium, rhenium, palladium, tungsten, nitinol and the like, as well as alloys of these metals; or radiolucent fibers or polymers such as Dacron (polyester), fluoropolymers, nylon, or even silk; or various combinations of metals and polymers.

7. The method for delivering the device of claim 1 to a target location within a mammalian body comprising:
adhering to and growing functional cells on said cell-composed device;
delivering said cell-composed device to targeted area;
releasing and placing said cell-composed device to said targeted area;
allowing said cell-composed device home in and engraft into said targeted area.

8. The method of claim 7, wherein the cell-composed device is introduced via a catheter based system.

9. A method of curing heart disease comprising:
adhering to and growing functional cells on biocompatible medical device;
introducing one or more said cell-composed device(s) via a catheter based system;
delivering said device(s) via minimally invasive intracornonary approach to the targeted area;
releasing and placing said device(s) into said targeted area;
allowing functional cells from said device(s) to home in and engraft to the zone of the targeted area at an optimal breadth and depth, in order to regenerate infarcted, scarred or non-functioning myocardial tissue into functioning muscle (“myogenesis”), also to create growth and proliferation of new blood vessel (“angiogenesis”) in said targeted area.

10. A three-dimensional cell composite comprising:
functional cells; and
cell culture medium.

11. The three-dimensional cell composite of claim 10, wherein the functional cells and the cell culture medium are compatible with the target location of a mammalian body.

12. The three-dimensional cell composite of claim 10, wherein the functional cells are selected from the group consisting of stem cells and progenitor cells such as human embryonic stem cells (hES); human embryonic germ cells (hEG); human embryonic carcinomas cells (hEC); adult stem cells (hS) originated from tissues including but not limited to bone marrow, peripheral blood, skeletal muscle; cornea and retina, brain, dental pulp, liver, gastrointestinal tract lining, adipose (fat) tissues and pancreas; or genetic or chemical engineered cells; or multipotent adult progenitor cells that can differentiated into cardiomyocyte and/or endothelial cells.

13. The three-dimensional cell composite of claim 10, wherein cell culture medium are selected from group consisting of various growth factors, enzymes, small molecules and other natural occurring and/or synthetic biocompatible, biodegradable materials useful for cell growth, differentiation and migration. In addition to growth factors and genetic material useful for cell growth, differentiation and migration, molecules such as rapamycin or FK560 may be added to help avoid immunorejection.

14. The method for delivering the device of claim 10 to a target location within a mammalian body comprising:
adhering and growing functional cells in said three-dimensional cell composite;
delivering said three-dimensional cell composite to targeted area;
allowing said three-dimensional cell composite flows into said targeted area.

15. The method of claim 14, wherein the three-dimensional cell composite is introduced via a catheter based system.

16. A method of curing heart disease comprising:
adhering and growing functional cells in biocompatible three-dimensional composite;
delivering said three-dimensional cell composite to targeted area;
allowing said three-dimensional cell composite flows into said targeted area, to home in and engraft to the zone of the targeted area at an optimal breadth and depth, in order to regenerate infarcted, scarred or non-functioning myocardial tissue into functioning muscle (“myogenesis”), also to create growth and proliferation of new blood vessel (“angiogenesis”) in said targeted area.
